(19) United States
(12) Patent Application Publication
(10) Pub. No.: US 2025/0359998 A1
(43) Pub. Date: Nov. 27, 2025

(54) APPARATUS, SYSTEM AND METHOD FOR ASSISTING A PATH OF MOTION

(71) Applicant: New York Society for the Relief of the Ruptured and Crippled, maintaining the Hospital for Special, New York, NY (US)

(72) Inventors: Lawrence Gulotta, New York, NY (US); Andreas Kontaxis, New York, NY (US)

(73) Assignee: New York Society for the Relief of the Ruptured and Crippled, maintaining the Hospital for Special, New York, NY (US)

(21) Appl. No.: 18/876,489

(22) PCT Filed: Jun. 28, 2023
(86) PCT No.: PCT/US2023/069283

§ 371 Date: Dec. 18, 2024

Related U.S. Application Data
(60) Provisional application No. 63/356,463, filed on Jun. 28, 2022.

(51) Int. Cl. A61F 2/40; A61F 2/30; A61F 2/38

(52) U.S. Cl. CPC A61F 2/4014; A61F 2/3804; A61F 2/4081; A61F 2002/30079; A61F 2002/30649; A61F 2002/3831

(57) ABSTRACT

A prosthetic joint (200) for assisting a path of motion that includes a first component (118) including a first magnet (130a, 130b, 130c); a second component (124) engaging with the first component, the second component including a second magnet (126), and wherein the second magnet is located with respect to the first magnet so as to be in magnetic attraction when the first component and/or the second component is moved along the path of motion.

### BACKGROUND OF THE DISCLOSURE

The subject disclosure relates generally to the field of medical device implants.

Surgery involving insertion of a joint prosthesis often disrupts muscles and/or nerves rendering certain motions about the joint difficult to execute. For example, patients rehabilitating from muscle tears and surgeries about a joint often experience difficulty in externally rotating about that joint. In the case of the shoulder, patients with rotator cuff tears that, for example, involve the infraspinatus and teres minor muscles can experience significant weakness with external rotation. Reverse shoulder arthroplasty (RSA) alone does not improve this functional deficit. Surgeons have tried adding a latissimus dorsi transfer to RSA subjects in these situations to help improve external rotation. While this procedure has allowed some patients to obtain modest improvements in function, it adds complexity to the procedure resulting in increased time in surgery, potential for complications and unpredictable results, and still does not adequately overcome external rotation weakness in all circumstances.

In another example, patients rehabilitating from insertion of an elbow prosthesis often experience difficulty in flexing or extending the elbow due to pre-operative or operative disruption of muscle and/or nerve.

Therefore, there remains a need for a mechanical, or implant-related solution to assist with certain motions about a joint such as, for example, flexion, extension, and/or external rotation.

### SUMMARY OF THE DISCLOSURE

Exemplary embodiments of the disclosure incorporate magnets that are positioned about a joint to provide an attractive magnetic force to facilitate movement about the joint (e.g., a prosthetic joint). For example, the systems and methods of the subject disclosure can be used to provide assistance in externally rotating about the joint, such as a prosthetic joint (e.g., a prosthetic joint provided pursuant to a reverse shoulder arthroplasty (RSA) or total shoulder arthroplasty (TSA)). In certain exemplary embodiments, the subject disclosure provides for the use of magnets to create an attractive force in the back (posterior) of the shoulder to help patients achieve external rotation following RSA or TSA.

The present disclosure provides a prosthetic joint for assisting a path of motion characterized by:
a first component including:
an articular end,
a stem end, and
a first magnet positioned about the articular end of the first component; and
a second component including:
an articular end,
a stem end, and
a second magnet positioned about the articular end of the second component;
wherein the second magnet is located with respect to the first magnet such that magnetic attraction will increase as at least one of the first magnet and the second magnet move along the path of motion.

In an embodiment, the prosthetic joint is a prosthetic shoulder joint, the first component is a prosthetic glenoid socket, and the second component is a prosthetic humerus. In embodiments, the path of motion is external rotation. In further embodiments, the first magnet and the second magnet are substantially not in magnetic attraction when the prosthetic joint is in a neutral rotation position.

In an embodiment, the first component is a glenosphere and the second component is a prosthetic humerus that includes a prosthetic cup insert designed to articulate with the glenosphere.

One exemplary embodiment of the subject disclosure provides a prosthetic joint for assisting external rotation about the shoulder that includes a first component including a first magnet, and a second component engaging with the first component, the second component including a second magnet. The second magnet is located with respect to the first magnet so as to be in magnetic attraction when the first component and/or the second component is moved along an external rotational path to be placed in an external rotation position.

In an exemplary embodiment, the first magnet and the second magnetic are substantially not in magnetic attraction when the joint is in a neutral position. In an exemplary embodiment, the first component including a first magnet is a glenoid socket (e.g., a prosthetic glenoid socket) and the second component including a second magnet is a humerus component (e.g. a prosthetic humerus that terminates with a prosthetic ball joint). In another exemplary embodiment, the first component including a first magnet is a glenosphere and the second component including a second magnet is a humerus component (e.g., a prosthetic humerus that includes a prosthetic cup insert adapted to articulate with the glenosphere).

One exemplary embodiment of the subject disclosure provides a Reverse Shoulder Arthroplasty (RSA) system for a subject that includes a humeral stem comprising a tray, and a cup insert, and a glenosphere. The tray includes at least one tray insert magnet and/or the cup insert includes at least one cup insert magnet; and the glenosphere includes at least one glenosphere magnet. The (a) cup insert magnet and/or tray insert magnet and (b) glenosphere magnet are each positioned, upon rotating the cup insert along an external rotation path, to be in magnetic attraction along an articulating surface between the cup insert and the glenosphere. In an exemplary embodiment, the at least one cup insert magnet or at least one or tray insert magnet and (b) the at least one glenosphere magnet are substantially not in magnetic attraction in a neutral rotation position.

The present disclosure provides a prosthetic joint, wherein the first component is a humeral stem comprising a tray and a cup insert, the cup insert including at least one cup insert magnet and/or the tray including at least one tray insert magnet; wherein the second component is a glenosphere comprising at least one glenosphere magnet; and wherein the path of motion is an external rotation path, (a) the at least one cup insert magnet or at least one tray insert magnet, and (b) the at least one glenosphere magnet, are each positioned to be in magnetic attraction upon rotating the cup insert along the external rotation path.

In embodiments, (a) the at least one cup insert magnet or at least one tray insert magnet and (b) the at least one glenosphere magnet are substantially not in magnetic attraction in a neutral rotation position. In embodiments, the prosthetic joint for assisting a path of motion the cup insert comprises a plurality of cup insert magnets. In embodiments, the glenosphere comprises a plurality of glenosphere magnets. In embodiments, the at least one glenosphere magnet is positioned in a posterior location of the glenosphere for positioning within a posterior location within a subject. In embodiments of the disclosure, the at least one glenosphere magnet is positioned at or near a base of the glenosphere. In embodiments of the disclosure, the glenosphere comprises a magnet extending toward a center of the glenosphere.

Embodiments of the disclosure may further comprise: one or more sensors to measure a magnetic field associated with the cup insert magnet, the tray insert magnet and/or the glenosphere magnet; and a controller to control a magnetic field provided by the cup insert magnet, the tray insert magnet and/or the glenosphere magnet; further comprising a user interface to interface with the controller, wherein the user interface includes a user display to display measured results from the one or more sensors and/or an indication of a setting for the controller.

In an exemplary embodiment, the glenosphere comprises a plurality of glenosphere magnets and/or the cup insert comprises a plurality of cup insert magnets. In an exemplary embodiment, the glenosphere magnet is positioned in a posterior location with respect to the subject, and/or the glenosphere magnet is positioned at or near a base of the glenosphere. In an exemplary embodiment, the magnets are permanent magnets. In an alternative embodiment, the magnets have an applied magnetic field (such as, e.g., upon the application of an electrical current).

Another exemplary embodiment of the subject disclosure provides a Total Shoulder Arthroplasty (TSA) system that includes a humeral stem comprising a ball joint, the ball joint including at least one ball joint magnet. The TSA system further includes a glenoid socket comprising at least one glenoid socket magnet. The ball joint magnet and the glenoid socket magnet are positioned, upon rotating the ball joint along an external rotation path, to be in magnetic attraction along an articulating surface between the ball joint and the glenoid socket. In an exemplary embodiment, the at least one ball joint magnet and the at least one glenoid socket magnet are substantially not in magnetic attraction in a neutral rotation position.

In an embodiment of the disclosure, the prosthetic joint is a prosthetic elbow joint, the first component is a prosthetic humerus, and the second component is a prosthetic ulnar. In another embodiment, the prosthetic joint is a prosthetic elbow, and the first component including a first magnet is a humeral component and the second component including a second magnet is an ulnar component. In an embodiment, the ulnar component second magnet is located with respect to the humeral component first magnet so as to be in magnetic attraction when the humeral component and/or the ulnar component is moved along an elbow extension path to place the elbow in an extension position. In a further embodiment, the ulnar component second magnet is located with respect to the humeral component first magnet so as to be in magnetic attraction when the humeral component and/or the ulnar component is moved along an elbow flexion path to place the elbow in a flexion position.

An embodiment of the disclosure provides a total elbow arthroplasty system that includes a humeral component including at least one magnet and an ulnar component including at least one magnet. The humeral component including at least one magnet and the ulnar component including at least one magnet are positioned to be in magnetic attraction upon following an extension path to place the elbow in an extended position. In an embodiment, the at least one humeral component magnet and the at least one ulnar component magnet are substantially not in magnetic attraction when the elbow is in a neutral position.

A further embodiment of the disclosure provides a total elbow arthroplasty system that includes a humeral component including at least one magnet and an ulnar component including at least one magnet wherein the humeral component at least one magnet and the ulnar component at least one magnet are positioned to be in magnetic attraction upon following a flexion path to place the elbow in a flexion position. In an embodiment, the at least one humeral component magnet and the at least one ulnar component magnet are substantially not in magnetic attraction when the elbow is in a neutral position. In an embodiment, the path of motion is extension. In another embodiment, the path of motion is flexion.

In an exemplary embodiment, the system further includes one or more sensors to measure a magnetic field associated with one or more magnets of the subject disclosure (e.g., the cup insert magnet and/or the glenosphere magnet); a controller to control a magnetic field provided by the magnets (e.g., the cup insert magnet and/or the glenosphere magnet); and a user interface to interface with the controller. In an exemplary embodiment, the user interface includes a user display to display measured results from the one or more sensors and/or an indication of a setting for the controller.

### BRIEF DESCRIPTION OF THE SEVERAL VIEWS OF THE DRAWINGS

The following detailed description of exemplary embodiments of the subject disclosure will be better understood when read in conjunction with the appended drawings. For the purpose of illustrating the present disclosure, there is shown in the drawings exemplary embodiments. It should be understood, however, that the subject application is not limited to the precise arrangements and instrumentalities shown.

FIG. 1 depicts the various range of motions that a shoulder undergoes during daily activities;

FIGS. 2A and 2B are posterior views of a Reverse Shoulder Arthroplasty (RSA) system;

FIGS. 3A, 3B and 3C are perspective views of a glenosphere (3A, 3B) and humeral components (3C) of a reverse shoulder arthroplasty system according to an exemplary embodiment of the subject disclosure;

FIGS. 3D and 3E are perspective views of a glenosphere (3D) and humeral components (3E) according to another exemplary embodiment of the subject disclosure;

FIG. 3F is a perspective view of humeral components according to yet another exemplary embodiment of the subject disclosure;

FIG. 4 is a perspective view of the exemplary embodiment of FIGS. 3A-3C as implanted in a subject;

FIGS. 5A and 5B are perspective views of the exemplary embodiment of FIGS. 3A-3C as implanted in a subject in a neutral rotation position and an external rotation position;

FIG. 6 is a posterior view of a total shoulder arthroplasty (TSA) system according to an exemplary embodiment of the subject disclosure;

FIG. 7 is a schematic of a control system according to an exemplary embodiment of the subject disclosure;

FIG. 8 shows a prosthesis inserted into an elbow joint;

FIG. 9 is a perspective view of an exemplary path of motion assist system for elbow extension; and

FIG. 10 is a perspective view of an exemplary path of motion assist system for elbow flexion.

### DETAILED DESCRIPTION

Reference will now be made in detail to an exemplary embodiment of the subject disclosure illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as upper, lower, top, bottom, above, below and diagonal, are used with respect to the accompanying drawings. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. For example, magnets are substantially not in magnetic attraction when there is no magnetic attraction present, or such minimal magnetic attraction that, though measurable, are not sufficient to exert a physiological affect or influence on the subject so as not to be noticeable by the subject.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the subject disclosure can be practiced without one or more of the specific features or advantages of a particular exemplary embodiment. In other instances, additional features and advantages may be recognized in certain embodiments that may not be present in all exemplary embodiments of the present disclosure.

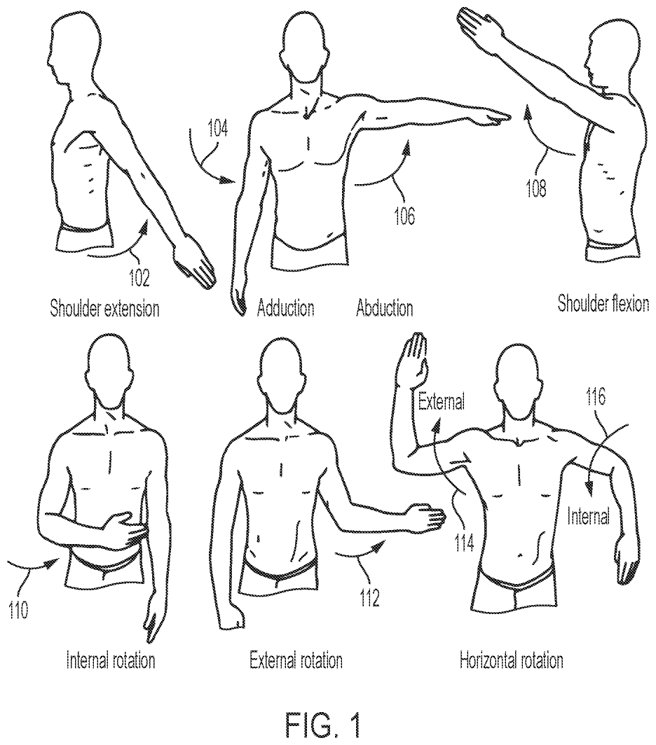

Referring now to the drawings, FIG. 1 discloses various ranges of motion that a subject exhibits with the shoulder that are attendant to performing daily life functions. These include shoulder extension 102, adduction 104, abduction 106 and shoulder flexion 108. The shoulder is also rotated regularly, including internal rotation 110 and external rotation 112 at a relatively low degree of abduction. Horizontal external rotation 114 and horizontal internal rotation 116 also occur at higher degrees of shoulder abduction, as shown in the bottom right panel of FIG. 1.

In exemplary embodiments, the subject disclosure incorporates a magnetic field (e.g., magnets) about a joint to facilitate external rotation about that joint. For example, after undergoing a reverse shoulder arthroplasty, subjects often experience difficulty externally rotating their shoulder, such as achieving movements 112 and 114 as shown in FIG. 1; and in exemplary embodiments, the instantly disclosed systems and methods assist the subject in external rotation 112 and/or external horizontal rotation 114. As used herein, “the external rotation position” refers to the position of the head of the humerus (and in the context of an RSA, the position of the humeral stem and cup insert) about the glenoid cavity (or glenosphere in the context of an RSA) after movements 112 and 114 in FIG. 1 are achieved.

While the subject disclosure will be described below in the context of a reverse shoulder arthroplasty and total elbow arthroplasty, it is understood, however, that the subject disclosure can equally be applied to other shoulder and elbow procedures (e.g., Total Shoulder Arthroplasty), and to anatomical (i.e., non-prosthetic) and prosthetic joints besides the shoulder and elbow (e.g., knee and hip joints).

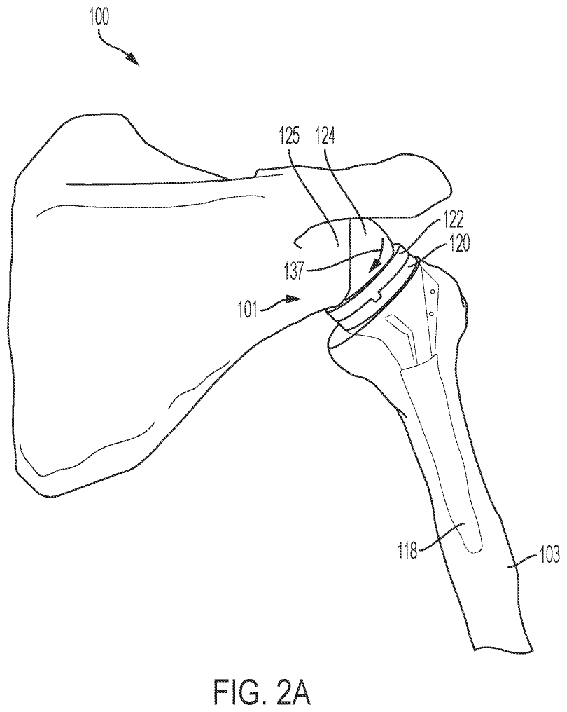

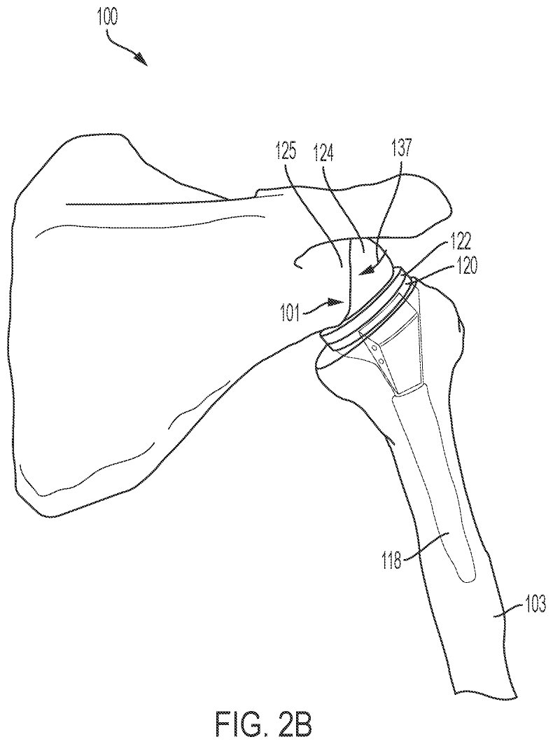

FIGS. 2A and 2B illustrate a posterior view of a reverse should arthroplasty (RSA) system 100 implanted in a subject's right shoulder 101 (as modeled). In FIG. 2A, the subject is holding an object in front of them. In FIG. 2B, the subject has externally rotated their right humerus 103 about their shoulder 101 so as to position the object behind them, while maintaining a relatively constant degree of abduction.

The components of an RSA system 100 are shown in FIGS. 2A and 2B. The RSA incorporates a humeral stem 118 that terminates at a proximal end of the stem with a tray 120. A cup insert 122, which can be made of a biocompatible component (e.g., polyethylene) is engaged with the tray 120. The cup insert 122 provides an articulating surface, articulating with a glenosphere 124. The glenosphere 124 is secured to a baseplate (not shown) that is secured (e.g., screwed) into the native glenoid 125. FIG. 2A depicts the subject in a standard resting position and in a neutral rotational position (e.g., position in which the subject's arm appends from their shoulder as generally dictated by gravity). FIG. 2B depicts the subject in a position in which the subject has externally rotated their shoulder. When the subject externally rotates their shoulder, the cup insert 122 forms an articulating surface with the glenosphere 124 along an external rotation path 137.

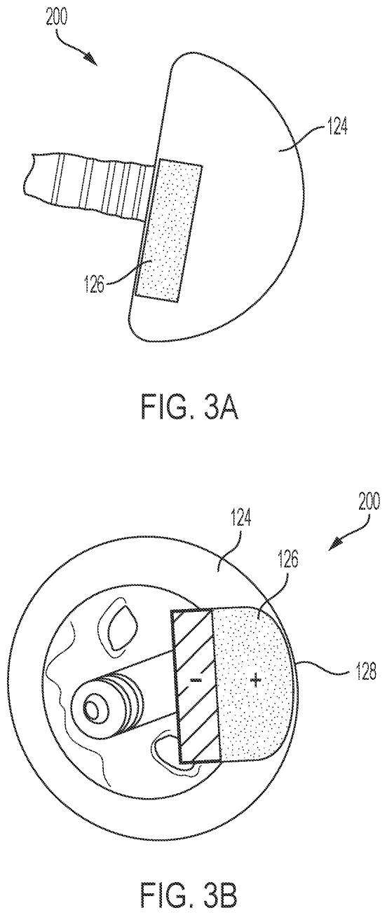

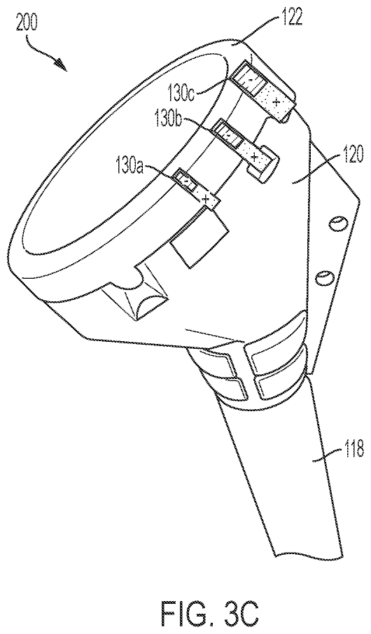

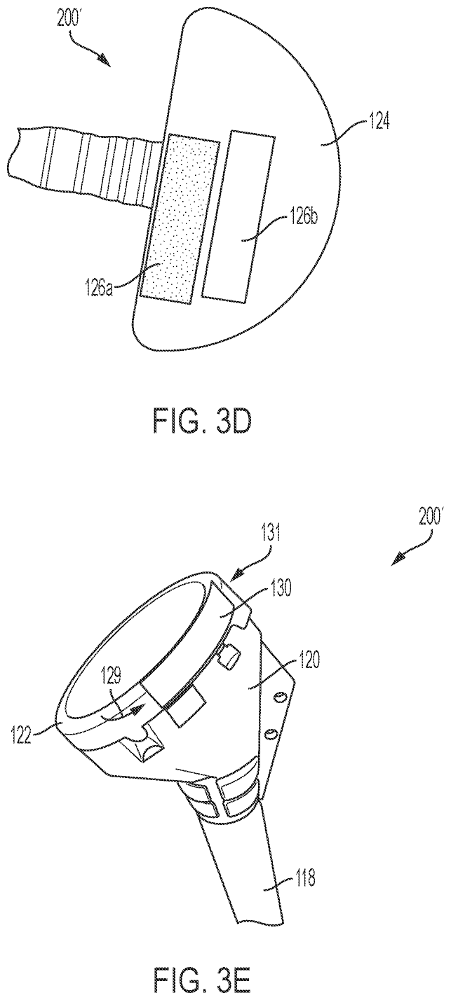

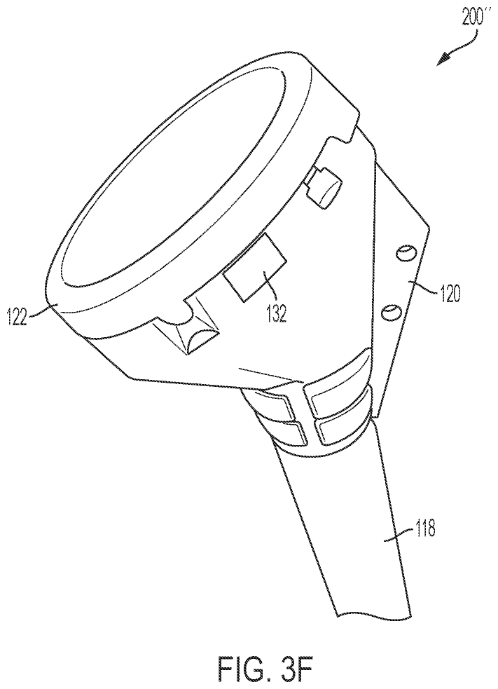

FIGS. 3A-3C illustrate a system 200 according to an exemplary embodiment in which the glenosphere 124 and cup insert 122 components of the RSA system 100 include magnets.

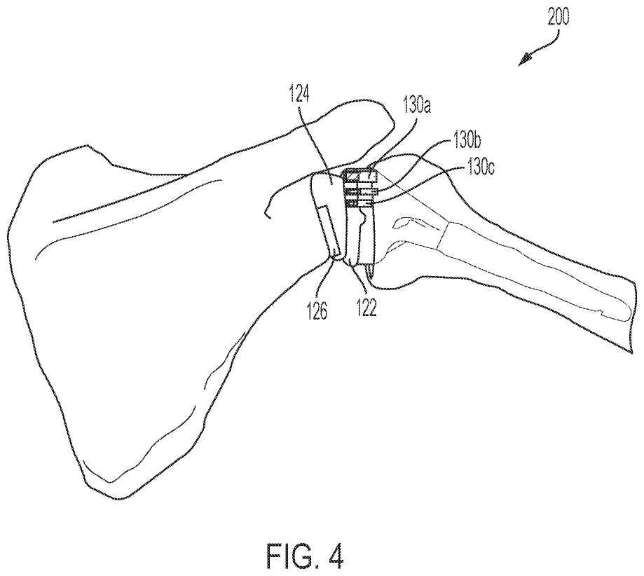

The genlosphere 124 is configured as best shown in FIGS. 3A and 3B The glensphere includes a glenosphere magnet 126 located at or near the outer base of the glenosphere 124, i.e., at or near the portion where the glenosphere is joined to a glenoid and at or near the outer surface of the glenosphere. FIG. 3A illustrates a posterior view of the glenosphere 124. As shown in FIG. 3A, when the glenosphere is implanted in a subject, the glenosphere magnet 126 is located in a posterior position with respect to the subject (see also, e.g., FIG. 4).

In this exemplary embodiment, a positive pole of the glenosphere magnet 126 is oriented outward, with the magnet's outer perimetry located near the outer perimetry of the glenosphere 124, as shown in schematic form in FIG. 3B. Alternatively, the glenosphere magnet 126 can be disposed such that it forms the outer perimetry of the glenosphere itself. The negative pole is oriented inward, toward the center of the glenosphere.

The number and placement of magnets about the glenosphere can vary so as to exert an attractive magnetic force along a surface of the glenosphere that articulates with the cup insert at a location that occurs when a subject, via movement of the humerus, places the shoulder along an external rotation path. FIG. 3D discloses the glenosphere 124 according to an alternative embodiment 200′, in which a plurality of glenosphere magnets 126a, 126b is provided in the place of a single glenosphere magnet 126, as shown in FIG. 3A. While the number and exact placement of the plurality of glenosphere magnets 126a, 126b can vary, in this exemplary embodiment, the glenosphere magnet 126b is disposed toward the center of the glenosphere. Also, in this exemplary embodiment, the glenosphere magnet 126b disposed, with respect to the first glenosphere magnet 126a, is to extend into a superior position on the glenosphere. In this exemplary embodiment, glenosphere magnet 126a has a higher magnetic strength than glenosphere magnet 126b, though alternatively, for example, magnets of equal strength can be provided. Alternatively expressed, the plurality of glenosphere magnets be configured to have varying levels or different levels of magnetic strength.

shown in FIG. 3E, the plurality of cup insert magnets 130a, 130b 130c can be replaced with a single cup insert magnet 130 located at or near an articulating surface of the cup insert 122 according to alternative embodiment 200′. In this exemplary embodiment, the magnetic strength of the magnetic increases (e.g., in a linear or an exponential manner) across its length, i.e., the magnetic strength increases from end 129 to end 131, with a maximum magnetic strength at end 131. Alternatively, the cup insert magnet 130 can have a constant magnetic strength across its entire length, i.e., a constant magnetic strength from end 129 to end 131 of the cup insert magnet 130.

As shown in FIG. 3F, as an alternative embodiment 200″, a tray insert magnet 132 is incorporated into the tray 120 to provide a magnetic field about the tray. In this exemplary embodiment, the tray insert magnet 132 is provided in place of the cup insert magnets, though in alternative embodiments, both the tray insert magnet and cup insert magnets (e.g., cup insert magnets 130 or 130a, 130b, 130c) can both be provided to collectively exert a magnetic attraction on the glenosphere magnet. The tray insert magnet 132 is located about the tray 120 such that it is in sufficient proximity to the location of the glenosphere magnet upon rotating the humeral stem (and humerus) along an external rotation path so as to exert magnetic attraction with the glenosphere magnet. For example, the tray insert magnet can be positioned adjacent a superior end or superior surface of the tray.

It should be understood that the mating components of embodiments 200 and 200′ and 200″, as discussed above, are completely interchangeable. More particularly, for example, the glenosphere of embodiment 200 (see FIG. 3A) can be used with the humeral stem components of embodiment 200′ or 200″ (see FIG. 3E and FIG. 3F), and the glenosphere of embodiment 200′ (see FIG. 3D) can be used with humeral stem components of embodiment 200 or 200″ (see FIG. 3C and FIG. 3F).

If the positioning of the glenosphere magnet is not evident based on visual inspection (e.g., such as when the glenosphere magnet is located near the outer perimetry of the glenosphere, but not forming the outer surface itself), then the glenosphere can be marked with indicia 128 to visually indicate the positioning of the glenosphere magnet about the glenosphere. Such indicia can assist a surgeon in properly implanting and positioning the glenosphere so as to provide external rotation assistance along the external rotation path.

Referring back to FIG. 3C as an example, which depicts the humeral stem 118, tray 120 and cup insert 122 as modified according to this exemplary system 200, the cup insert 122 includes a plurality of cup insert magnets 130a, 130b and 130c. Though other configurations can be provided, in this exemplary embodiment, the cup insert magnets have different strengths: 130c having a higher magnetic strength than 130b, and 130b having a higher magnetic strength than 130a. The plurality of cup insert magnets 130a, 130b and 130c are spaced apart and located along the outer rim of the cup insert 122. In this exemplary embodiment, the plurality of cup insert magnets 130a, 130b and 130c are positioned such that the negative pole is positioned closest to the articulating surfaces of the cup insert 122, with the positive pole being disposed away from the articulating surfaces of the cup insert.

FIG. 4 depicts a posterior view of the system 200 as implanted in a subject, in which the subject has neutral shoulder rotational position. As implanted in the subject, the glenosphere magnet 126 is located along a posterior and slightly inferior portion of glenosphere. In this exemplary embodiment, the glenosphere magnet 126 is a relatively strong magnet, e.g., one having at least 1500 Gauss.

The plurality of cup insert magnets 130a, 130b and 130c are positioned near articulating surfaces of the cup insert 122 where it engages with the glenosphere 124 having its magnet located along a posterior and slightly inferior portion of glenosphere. In the neutral rotational position shown in FIG. 4, however, the plurality of cup insert magnets 130a, 130b, and 130 are spaced apart from the glenosphere magnet 126 so as not to be influenced by the glenosphere magnet 126. Stated differently, in certain exemplary embodiments, there is no magnetic attraction (or repulsion) imparted upon the subject when the subject's shoulder is anatomically placed in a neutral rotational position (e.g., in a standard resting position in which a subject's arm append from their shoulder as generally dictated by gravity) owing to the positioning of the respective components/implants magnets when implanted in a subject.

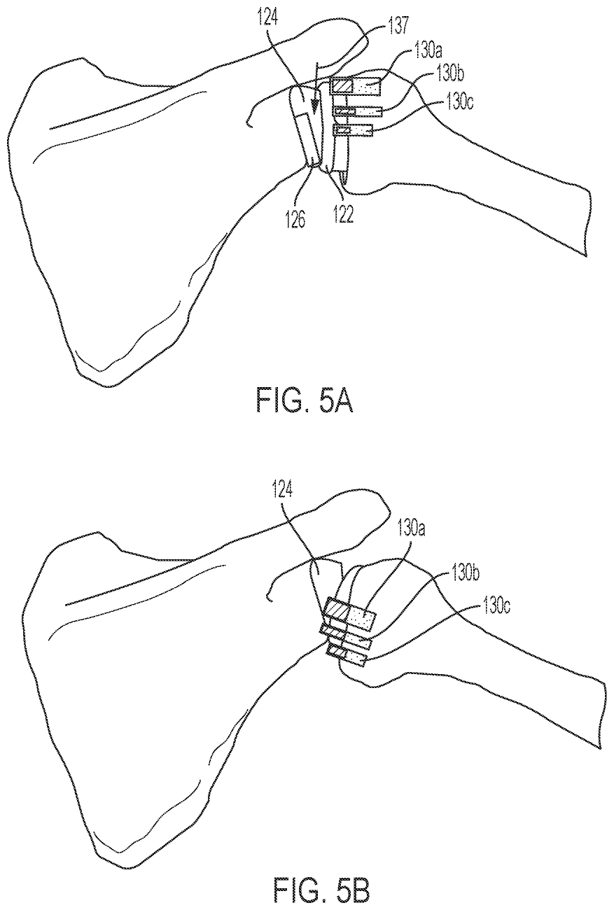

More particularly, as shown in FIG. 5A, in a neutral rotational position the cup insert 122 articulates laterally from the base of the glenosphere 124, with at least cup insert magnet 130a being located superior to the glenosphere magnet 126. When the shoulder of the subject is externally rotated (i.e., when the cup insert 122 is rotated by movement of the humerus), as shown in FIG. 5B, the cup insert 122 forms an articulating surface with the glenosphere 124 that is inferior and medial, as compared to the articulating surface in the neutral rotation position. As a result, when the humerus is moved along external rotation path 137 to be placed in the external rotation position, cup insert magnets 130a, 130b and 130c overlap with the glenosphere magnet and are in magnetic attraction. This in turn facilitates external rotation of the shoulder. It is noted that the position of either the glenosphere magnet 126 and/or the cup insert magnets 130a, 130b, 130c can be adjusted so long as the two are placed in magnetic attraction along the external rotation path 137 from the neutral rotation position shown in FIG. 5A. To be placed in magnetic attraction as used herein means that an electromagnetic force is in play causing objects to be drawn together.

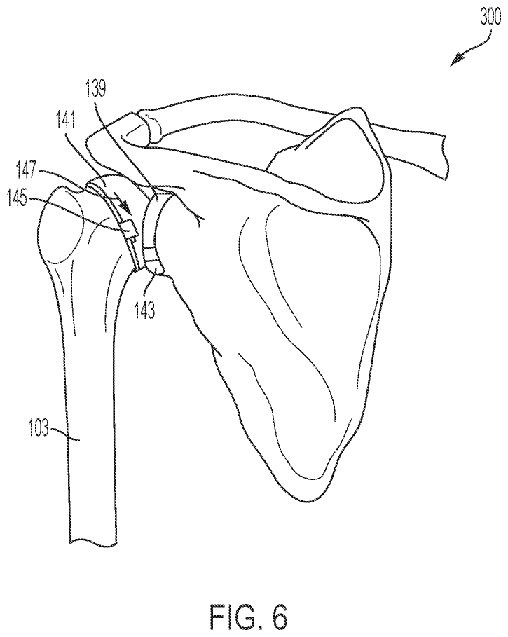

As noted, while the present subject matter is described above in the context of a reverse shoulder arthroplasty and a total elbow arthroplasty, it is understood, however, that it can equally be applied to other shoulder procedures (Total Shoulder Arthroplasty), and to anatomical (i.e., non-prosthetic) and prosthetic joints besides the shoulder and elbow (e.g., knee and hip joints). For example, FIG. 6 depicts and exemplary embodiment of the subject disclosure in the context of a Total Shoulder Arthroplasty (TSA). FIG. 6 depicts a posterior view of a subject's left shoulder that includes a prosthetic joint 300 of the subject disclosure in a neutral rotation position.

A TSA includes a prosthetic glenoid socket 139, which is generally incorporated into the native glenoid cavity and constructed of a material suitable for forming an articulating surface (e.g., polythethylene). The humerus 103 terminates with a ball joint 141, which articulates with the glenoid socket 139. The glenoid socket is provided with a glenoid socket magnet 143, which is located in an inferior location of the glenoid socket. The ball joint 141 is provided with a ball joint magnet 145 that is located about a posterior side of the ball joint, superior to glenoid socket magnet 143 when the subject is in a neutral rotation position, as shown in FIG. 6.

The polarities of the glenoid socket magnet 143 and ball joint magnet 145 are arranged to be in an attractive relationship when placed in sufficient proximity to each other. In this exemplary embodiment, the glenoid socket magnet 143 and ball joint magnet 145 are sufficiently spaced apart in the neutral rotation position so as to be substantially not in magnetic attraction. As the ball joint 141 is moved along an external rotation path 147, and placed in an external rotation position, the ball joint magnet 145 approaches the glenoid socket magnet 143 so as to be in magnetic attraction, thereby facilitating external rotation of the shoulder. As the ball joint 141 moves to achieve further external rotation, the ball joint magnet 145 eventually aligns with, or at least more closely approaches, the glenoid socket magnet 143 such that magnetic attraction is increased as the shoulder further externally rotates.

The magnets of the subject disclosure (e.g., magnets 126, 130a, 130b, 130c) can be a permanent magnet and/or can have a magnetic field applied thereto (e.g., via the application of electric current, such as via a controller and one or more sensors).

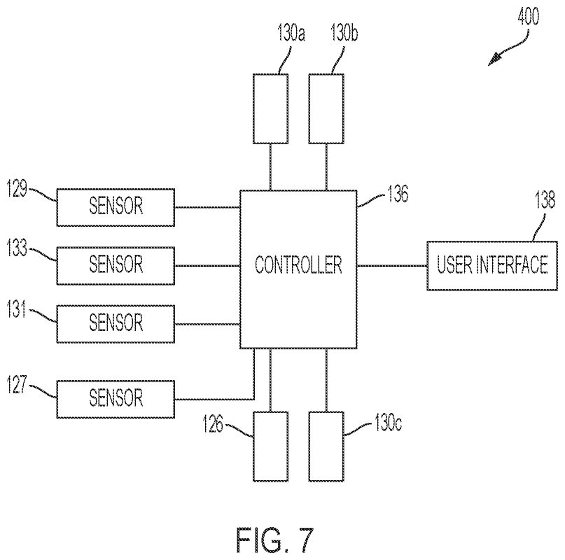

With reference to FIG. 7 and system 200, a controller 136 can optionally be provided as part of control system 400 to provide specific amounts of electric current to the magnets, and also to be in communication with one or more sensors (e.g., magnetic sensors) to measure a magnetic field associated with each magnet. While discussed above in connection with system 200, it is understood that the control system 400 can be applied to any of the instantly disclosed prosthetic joints.

More particularly, as shown in FIG. 7, a controller 136 can optionally be provided. The controller 136 is in electronic communication with the glenosphere magnet 126, and the plurality of cup insert magnets 130a, 130b, and 130c. The controller 136 is in communication with sensor 127, which measures a magnetic field associated with magnet 126, sensor 129, which measures a magnetic field associated with magnet 130a, sensor 131, which measures a magnetic field associated with magnet 130b and sensor 133, which measures a magnetic field associated with the magnet 130c. A user interface 138 is in communication with the controller 136, and allows an operator to input the desired settings to the controller and display sensed results from the sensors.

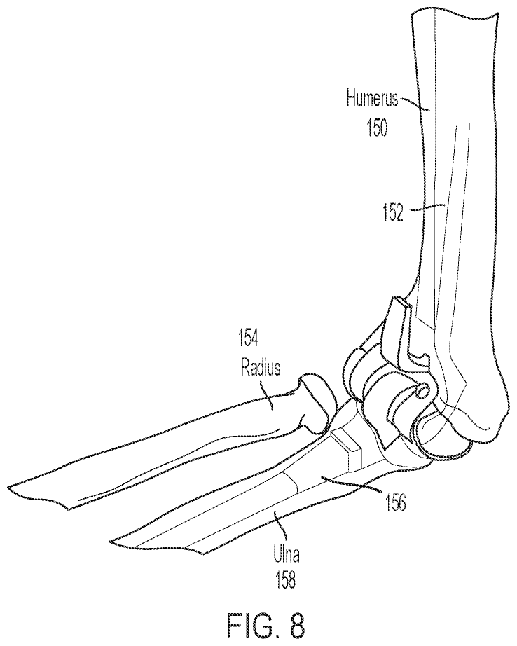

FIG. 8 shows a prosthetic elbow joint 500 wherein a humerus bone 150 has an inserted prosthetic humerus component 170 and an ulnar bone 158 has an inserted prosthetic ulnar component 158. The native radius bone 154 is also shown.

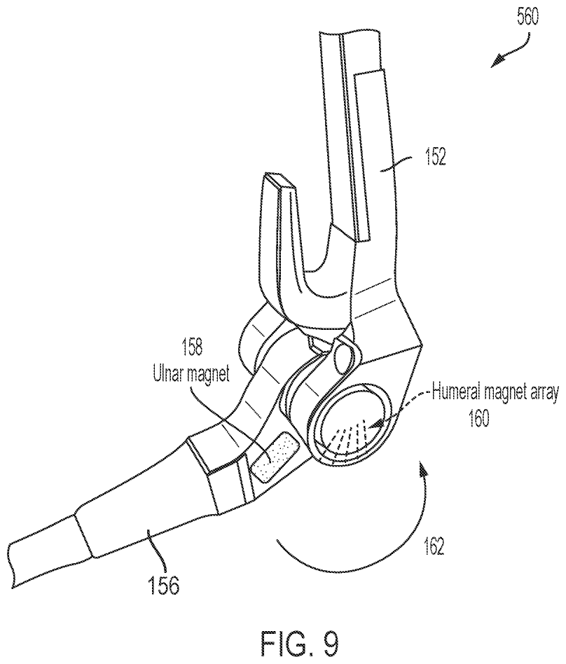

Referring to FIG. 9, the prosthetic elbow joint 500 includes a magnet array 160 in the prosthetic humerus component 152 and a magnet array 162 in the prosthetic ulnar component 172 configured to facilitate elbow extension 162.

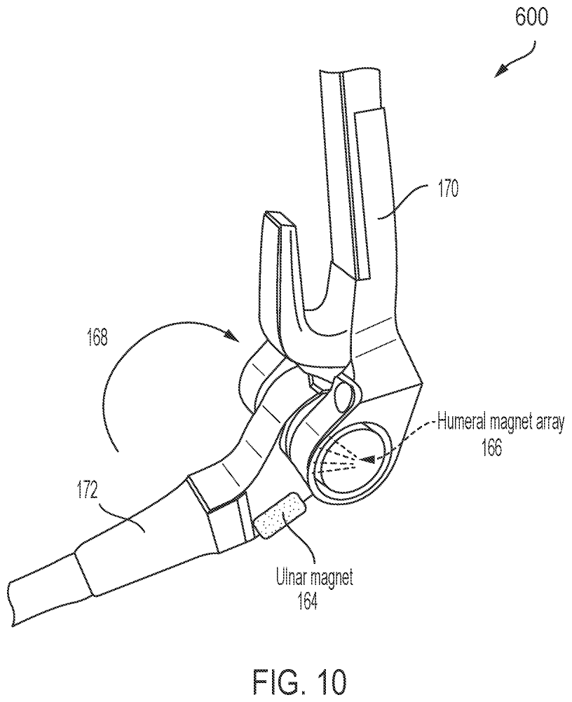

FIG. 10 shows an exemplary prosthetic elbow joint 600 having a magnet array 166 in a prosthetic humerus component 170 and a magnet array 164 in the prosthetic ulnar component 172 to facilitate elbow flexion 168.

The absolute and relative magnetic strengths of the magnets of the subject disclosure, (e.g., glenosphere magnet 126, and the plurality of cup insert magnets 130a, 130b and 130c) can be determined by one of ordinary skill in the art in view of, for example, the particular subject to be treated and the rotational assistance needed for that particular subject, e.g., the necessary rotation assistance required in order for the subject to comfortably (or more comfortably) achieve movements 112 and 114 as shown in FIG. 1. The magnets can be constructed of magnetic materials known in art, including neodymium, metal alloy, ceramic or rubberized magnetic material, and others as disclosed e.g., in U.S. Pat. No. 10,335,282, the disclosure of which is hereby incorporated by reference in its entirety.

In certain embodiments, the size, positioning, relative and/or absolute magnetic strengths of the magnets incorporated herein (e.g., the absolute and relative magnetic strengths of glenosphere magnet 126, and the plurality of cup insert magnets 130a, 130b and 130c) are specifically customized for the particular subject. In other embodiments, the size, positioning, relative and/or absolute magnetic strengths of the magnets incorporated herein (e.g., the absolute and relative magnetic strengths of glenosphere magnet 126, and the plurality of cup insert magnets 130a, 130b and 130c) are selected based on the average rotational assistance needed for a particular subject pool, or the entire subject pool.

For example, in certain embodiments, the instantly disclosed prosthetic joints are specifically designed for a pseudoparalysis or a pseudoparesis subject pool in which patients who have undergone a procedure (e.g., RSA or TSA) have particular trouble in achieving overhead motion, due to, for example, massive rotator cuff tears or rotator cuff atrophy. See, e.g., Pseudoparalysis and pseudoparesis of the shoulder, Obere Extremität 16:237-246 (2021), hereby incorporated by reference. Such subjects have particular difficulty in achieving, for example horizontal external motion (see motion 114 in FIG. 1), which the arm is held at a relatively higher degree of shoulder abduction. In such embodiments, for example, the size, strength and/or placement of magnets can be modified such that a substantial magnetic field of attraction (e.g., a magnetic field of attraction sufficient to affect physical movement) is achieved earlier in the external rotation path, and/or modified to a particularly assist motion 114 in FIG. 1 and external rotation movements when the arm is held at a high degree of shoulder abduction. In addition, or as an alternative, the magnets can be positioned to provide rotational assistance while the subject's shoulder is placed in a neutral rotation position, particularly when the humerus is elevated so as to provide further assistance in achieving horizontal external rotation.

The strength of the magnets can range, for example in exemplary embodiments, from about 0.25 gauss to 10,000 gauss, or from about 0.5 gauss to about 5000 gauss, or from about 1 gauss to about 2500 gauss, or from about 5 or 10 gauss to about 1500 or 2000 gauss. In exemplary embodiments the relative strength of the plurality of magnets, i.e., the ratio of the strength of the strongest magnetic in the plurality to the strength of the weakest magnet in the plurality (e.g., the ratio of the strength of cup insert magnet 130c to the strength of cup insert magnet 130a) can range from about 1.1 to about 5 or 10, or from about 1.25 to about 4, or from about 1.5 to about 3. These magnetic strengths, absolute and relative, are provided as examples only. In other embodiments, magnets of other relative and absolute strengths can be employed.

It will be appreciated by those skilled in the art that changes could be made to the exemplary embodiments described above without departing from the broad inventive concept thereof. It is to be understood, therefore, that this disclosure is not limited to the particular embodiments disclosed, but it is intended to cover modifications within the spirit and scope of the claims defined herein.

1. A prosthetic joint for assisting a path of motion, characterized by:
a first component including:
an articular end,
a stem end, and
a first magnet positioned about the articular end of the first component; and
a second component including:
an articular end,
a stem end, and
a second magnet positioned about the articular end of the second component;
wherein the second magnet is located with respect to the first magnet such that magnetic attraction will increase as at least one of the first magnet and the second magnet move along a path of motion.

2. The prosthetic joint for assisting a path of motion of claim 1, wherein the prosthetic joint is a prosthetic shoulder joint, the first component is a prosthetic glenoid socket, and the second component is a ball joint.

3. The prosthetic joint for assisting a path of motion of claim 2, wherein the path of motion is external rotation.

4. The prosthetic joint for assisting a path of motion of claim 1, wherein the first magnet and the second magnet are substantially not in magnetic attraction when the prosthetic joint is in a neutral rotation position.

5. The prosthetic joint for assisting a path of motion of claim 1, wherein the first component is a glenosphere and the second component is a prosthetic humerus that includes a prosthetic cup insert configured to articulate with the glenosphere.

6. The prosthetic joint for assisting a range of motion of claim 1, wherein the prosthetic joint is a prosthetic elbow joint, the first component is a prosthetic humerus, the first magnet is a humeral magnet, the second component is a prosthetic ulnar, and the second magnet is an ulnar magnet.

7. The prosthetic joint for assisting a path of motion of claim 6, wherein the path of motion is extension.

8. The prosthetic joint for assisting a path of motion of claim 6, wherein the path of motion is flexion.

9. The prosthetic joint for assisting a path of motion of claim 1, wherein the first component is a humeral stem comprising a tray and a cup insert, the cup insert including at least one cup insert magnet and/or the tray including at least one tray insert magnet;
wherein the second component is a glenosphere comprising at least one glenosphere magnet;
wherein the path of motion is an external rotation path; and
wherein (a) the at least one cup insert magnet or at least one tray insert magnet, and (b) the at least one glenosphere magnet, are each positioned to be in magnetic attraction upon rotating the cup insert along the external rotation path.

10. The prosthetic joint for assisting a path of motion of claim 9, wherein (a) the at least one cup insert magnet or at least one tray insert magnet, and (b) the at least one glenosphere magnet are substantially not in magnetic attraction in a neutral rotation position.

11. The prosthetic joint for assisting a path of motion of claim 9, wherein the cup insert comprises a plurality of cup insert magnets.

12. The prosthetic joint for assisting a path of motion of claim 9, wherein the glenosphere comprises a plurality of glenosphere magnets.

13. The prosthetic joint for assisting a path of motion of claim 9, wherein the at least one glenosphere magnet is positioned in a posterior location of the glenosphere for positioning within a posterior location within a subject.

14. The prosthetic joint for assisting a path of motion of claim 9, wherein the at least one glenosphere magnet is positioned at or near a base of the glenosphere.

15. The prosthetic joint for assisting a path of motion of claim 9, wherein the glenosphere comprises a magnet extending toward a center of the glenosphere.

16. The prosthetic joint for assisting a path of motion of claim 9, wherein the cup insert magnet and glenosphere magnet are permanent magnets.

17. The prosthetic joint for assisting a path of motion of claim 9, wherein the cup insert magnet and glenosphere magnetic have an applied magnetic field.

18. The prosthetic joint for assisting a path of motion of according to claim 9, further comprising:
one or more sensors to measure a magnetic field associated with the cup insert magnet, the tray insert magnet and/or the glenosphere magnet;
a controller to control a magnetic field provided by the cup insert magnet, the tray insert magnet and/or the glenosphere magnet; and
a user interface to interface with the controller, wherein the user interface includes a user display to display measured results from the one or more sensors and/or an indication of a setting for the controller.

19. The prosthetic joint for assisting a path of motion of according to claim 6, further comprising:
one or more sensors to measure a magnetic field associated with the humeral magnet and/or the ulnar magnet;
a controller to control a magnetic field provided by the humeral magnet and/or the ulnar magnet; and
a user interface to interface with the controller, wherein the user interface includes a user display to display measured results from the one or more sensors and/or an indication of a setting for the controller.
